(19) United States
(12) Patent Application Publication
(10) Pub. No.: US 2019/0241861 A1
(43) Pub. Date: Aug. 8, 2019

(54) BACTERIAL DETECTION SYSTEM

(71) Applicants: Karmel Alon Allison, Mountain View, CA (US); David James Allison, Mountain View, CA (US)

(72) Inventors: Karmel Alon Allison, Mountain View, CA (US); David James Allison, Mountain View, CA (US)

(21) Appl. No.: 15/887,975

(22) Filed: Feb. 2, 2018

(51) Int. Cl. C12M 1/34; C12Q 1/10; C12M 1/00; C12Q 1/06; C12Q 1/12; C12Q 1/14; B01L 3/00; C12N 1/20

(52) U.S. Cl. CPC C12M 41/36; C12Q 1/10; C12M 23/02; C12Q 1/06; C12Q 1/12; B01L 2300/0803; B01L 3/508; C12N 1/20; C12Q 2304/80; B01L 2300/0645; C12Q 1/14

(57) ABSTRACT

A method and device for detection of specific bacteria of interest by monitoring for chemical changes in media using electrical circuits. The present invention provides an inexpensive method to detect and measure particular bacteria of interest in samples, and is useful in particular as a means of monitoring for contamination in water sources.

### FIELD OF THE INVENTION

The present invention disclosed herein relates to a system for indirect electrochemical detection of bacteria in water or in a solution capable of supporting the growth of bacteria.

### BACKGROUND

Municipal and commercial water distribution facilities monitor several properties that can identify the quality of the water that they distribute. Such monitoring ensures that the water is potable at its source, throughout a distribution network, and at the point of delivery to the consumer. For example, water utilities continuously track water temperature, pH, dissolved oxygen, and conductivity.

A water quality parameter that is key to public health is bacterial level. However, it is not continuously monitored by water utilities. Water quality managers at utilities would be quick to acknowledge that this is a health concern, and that it would be beneficial to monitor for bacterial contamination, such as Escherichia or Enterococcus bacteria, continuously. However, a system to monitor for bacterial levels continuously and affordably does not currently exist in the marketplace.

Contaminating bacteria of interest include fecal coliform bacteria such as Escherichia coli. The presence of these coliform bacteria is an indicator of fecal contamination in a fresh water supply. Fecal contamination has multiple causes such as a break in a sewage pipe or insufficient treatment of surface water. Millions of people become sick each year due to exposure to water contaminated with fecal matter. The Environmental Protection Agency (EPA) thus requires that all water distribution facilities regularly monitor for the presence of coliform at multiple nodes across their distribution networks.

There is currently no cost-effective way to measure bacteria levels in water supplies continuously or remotely. Water is sampled manually within water distribution systems at specific points and at periodic intervals, and bacteria levels are determined using fluorescence or culturing tests. The collection and testing process is expensive, time-consuming, and produces a small amount of water quality data. For example, only one data point per month or quarter may be obtained due to the cost and labor of testing for bacteria. The low frequency of testing may allow bacterial contamination to be undetected and become widespread.

In US Patent Application 2005/0059105, Alocilja, E. C., et al. disclose a biosensor that is used to monitor impedance to rapidly detect bacterial pathogens in solution. The device in Alocilja, et al., describes a biosensor that directly binds a specific type of bacterium, using an interaction such as an antibody to the specific bacterium, and directly measures the impedance in electrical current across the biosensor components caused by bacteria bound to the biosensor. The biosensor in Alocilja, et al., is a relatively time consuming and expensive device to fabricate. The bacterial detection system described therein is intended to use laborious manual sampling methods. The biosensor of Alocilja, et al., is intended to be a handheld device and is not a cost effective means for continuous bacterial monitoring. The disclosure of Alocilja, et al., also does not provide an indirect means to monitor growth of bacteria that results in a secondary change in electrical properties of solution in which they are sampled.

In US Patent Application 2014/0182363, Potyrailo, R. A., et al. disclose a method using a sensor that employs a plurality of resonant circuits and a plurality of tuning elements to detect a spectrum of changes in impedance or changes in other environmental properties of a sample solution. However, Potyrailo, et al., do not disclose a method that is able to measure bacterial concentration from the spectrum of properties that it is used to analyze.

The disclosure of Ikemizu, M., et al., in US Patent Application 2016/0018391 describes a polymer-based sensor which has a three-dimensional structure that is complementary to a microorganism and which is capable of capturing the microorganism on the polymer. The captured microorganism creates a physical or electrical change, for example mass change or electrical conductance change that is detected by an electrode to which the polymer is bound. However, the sensor disclosed in Ikemizu, et al. is relatively expensive and the microorganism that is to be detected should be in a state of having an excessive electrical charge. The system of Ikemizu, et al. does not allow for an inexpensive sensor that uses a chemical change in a selective media to create a reliable bulk solution detection method as in the present invention disclosed herein.

In the disclosure of US Patent Application 2016/0238555, Kim, U., et al., describe a system that employs a relatively inexpensive and simple electrode sensor to perform electrochemical analysis of an aqueous solution. However, the disclosure of Kim, et al. is designed to detect small environmental contaminants which contaminants produce ionic changes in the test solution and limited to dissolved elements, namely arsenic, and is not able to detect specific bacterial species of interest.

In UK Patent Application GB2063911, Tanaka, M. et al., disclose a method for testing or screening for the sensitivity or resistance of bacteria to an antibiotic medicine. The method disclosed by Tanaka, et al. uses an inexpensive electrode sensor that can detect the change in oxidation-reduction in a growth medium in which the bacteria of interest has been incubated. The method of Tanaka, et al. requires that the user incubate the system with bacteria to be tested. It is only a pharmacologic screening tool that uses a known species of bacteria in a closed chamber, and cannot distinguish different species of bacteria. In contrast to the present invention, Tanaka, et al. do not employ bacterial selective media to propagate specific bacteria in a random water sample, which bacteria metabolize the media and create a physico-chemical change in the bacterial growth medium then in turn produce electrical changes related to bacterial concentration that are detected by the system. Tanaka, et al. also do not test sampled water quality as in the present invention.

The present invention provides an inexpensive, reliable and indirect means to test for specific bacterial concentration in a sampled solution without manual intervention. The present invention can monitor for bacterial concentration based on fundamental principles of bacterial growth and metabolism using selective media. The present invention represents a large divergence from current methods, which require manual collection, filtration, and culturing of bacteria. None of the existing methods are time-saving and cost-effective and allow for continuous, automated use in a water distribution system.

### SUMMARY OF THE INVENTION

The present invention utilizes a sensor to measure electrical property fluctuations that result from chemical changes in a water sample that has been incubated with a bacterial growth medium. The system also provides a means to selectively grow specific bacteria using specific media. The specific media is metabolized by the specific bacteria and results in chemical changes that in turn induce changes in electrical properties of the media.

The present invention also provides an inexpensive method to detect and measure bacteria in sampled water and a means to sample and collect water over a period of hours.

### BRIEF DESCRIPTION OF THE DRAWINGS

FIG. 1 illustrates a diagram of an electrical circuit, 100, to detect resistance changes in an incubation vessel in which bacterial inoculum will be cultured using the present invention. The circuit includes a voltage source, 101, connected to an electrode, 102, that in turn is connected to a sealable vessel, 103. A reference resistor, 104, is used to compare the resistivity changes in the sealable vessel, 103. An analog measurement device, 105, is included for obtaining measurements. The circuit terminates at a ground, 106.

FIG. 2 shows an overhead view of one embodiment of the sealable vessel. FIG. 2 shows two 3D-printed saucers, with the top saucer, 200, attached to the bottom saucer, 201.

FIG. 3 shows a cross-section of the same device (as indicated in FIG. 2) with the top saucer, 300, still shown sealed to the bottom saucer, 301. A one-way ingress valve, 302, allows for sample infusion such that sample flows over a filter within the vessel, 303. Excess liquid flows through the egress valve, 304. Two electrodes are exposed within the cavity of the vessel, 305 and 306.

FIG. 4 shows the underside of the same device, including notches for proper assembly.

FIG. 5 shows a plot of measurements collected from the device. The y-axis indicates resistance measured in ohms, and the x-axis indicates time in hours. Each line shows the change in resistance of the media when the indicated concentration of bacteria is incubated in the device.

### DETAILED DESCRIPTION OF THE INVENTION

The following descriptions are considered to be illustrative of the principles of the present invention and are not intended to be limiting. One of skill in the art will recognize and understand that there are suitable modifications and equivalents that may be used which fall within the scope of the invention described herein. The use of singular forms “a,” “an,” and “the” include plural references unless the context clearly requires otherwise. The embodiments are not limited to those illustrated in the drawings. It should also be understood that the drawings are not necessarily to scale. In certain instances, details may have been omitted that are not necessary for an understanding of the embodiments disclosed herein, for example, conventional fabrication and assembly.

The present invention is a novel sensor platform that can be used in situ in a water distribution system to monitor for the presence of total coliform bacteria, fecal coliform bacteria, or Escherichia coli (E. coli). The sensor consists of a water filtration system that allows for the collection of bacteria from a water stream over a period of 8 to 12 hours. The filter and any collected contaminants are subsequently incubated for 12 to 24 hours in selective growth media. Throughout the incubation period, the resistance of the media is monitored via electrodes placed at either end of the incubation vessel. If coliform bacteria are present, lactose in the media will be metabolized, increasing the pH of the media and decreasing the relative resistance. The change in the resistance of the media is algorithmically detected as a correlated indicator of the level of total coliform bacteria present in the water source of interest.

Coliform are a general term for Gram-negative bacteria of the Enterobacteriaceae family which are capable of fermenting lactose. Some coliform bacteria can also survive in high salt concentrations. Fecal coliform bacteria are a subset of coliform bacteria that are also capable of colonizing human intestines and being passed in feces. Examples of coliform that can survive in high constituent or bile salt concentrations are the genus Escherichia of which Escherichia coli (E. coli) is a species. Most species of E. coli are fecal coliform type.

Enterococci are a genus of Gram-positive bacteria. Enterococci are characterized by their ability to ferment lactose, to survive in a wide range of temperatures, and to grow even in the presence of low pH or high salt concentration.

The unique durability of Escherichia and Enterococci is indicative of their ability to colonize inhospitable environments, including the intestinal tracts of humans and animals. Fecal coliforms pass from the intestines to feces, and therefore serve as an indicator of the presence of fecal matter in water and food. They serve as an early warning of the potentially dangerous members of the family, including E. coli. As a result, testing for the presence of coliform bacteria is a requirement in all water distribution systems in the U.S., as mandated by the U.S. Environmental Protection Agency (EPA). One of the challenges of testing for coliform in water and food supplies is that other, non-threatening bacterial species are present. Existing impedance microbiology systems test only for total bacterial content of a sample, which would result in consistent false positive results when testing food or water supplies.

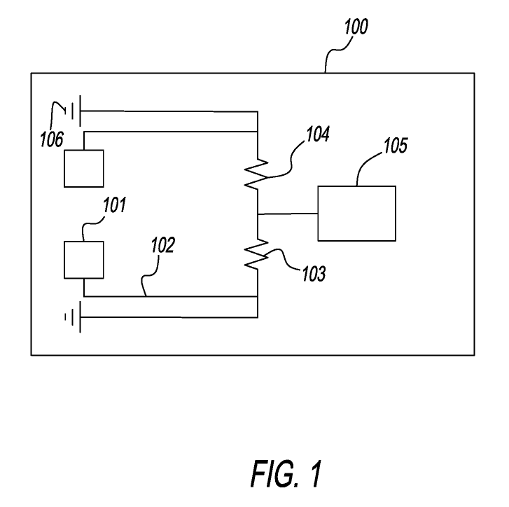

Referring to FIG. 1, a schematic of an embodiment of components of the present bacterial detection system, 100, is illustrated. The schematic of the present invention, 100, illustrates a voltage source of 5 volts, 101, connected to an electrode, 102, that in turn is connected to a sealable vessel, 103, an inoculum of a liquid to be tested for bacterial concentration is incubated in appropriate growth medium in the sealable vessel. FIG. 1 also illustrates a reference resistor, 104, that is used to compare the resistivity changes in the sealable vessel, 103. An analog measurement device, 105, for example, an ohmmeter and recorder is further illustrated. The circuit terminates at a ground, 106.

In one embodiment, the bacterial concentration measurement system of the present invention comprises a sealable vessel that has an electrical sensor that can produce measurements of a standard SI unit of electricity. The sealable vessel contains bacterial growth medium that is capable of supporting the growth of selected bacteria using selective growth constituents. The growth constituents are selected so that isolated species of bacteria, which are incubated in the sealable vessel, are able to metabolize the growth constituents into metabolites that alter the physico-chemical properties of the bacterial growth medium. The growth constituents are also selected so that the alterations or changes in the physico-chemical properties of the growth medium produce resulting and specific secondary electrical property changes in the growth medium. For example, the bacterial growth medium may contain lactose that can be metabolized by fecal coliform to produce lactic acid that changes the pH of the bacterial growth medium and which, in turn, results in a change in the electrical resistance of the growth medium during the incubation and growth of the fecal coliform bacteria. Not all species of bacteria can metabolize lactose. Thus, the use of a selective medium containing lactose will allow fecal coliform bacteria to produce lactic acid, and thus reduce the pH and decrease the resistance of the growth medium. The growth medium may further comprise bile constituents or bile salts to achieve enhanced selection and growth of members of the Escherichia genus or Enterococci genus of bacteria that can tolerate high concentrations of bile salts versus non-Escherichia or non-Enterococci bacteria. Not all species of bacteria or lactose-metabolizing bacteria can withstand high salt concentration. Thus the use of medium containing a high salt concentration derived from bile constituents or bile salts selects for fecal coliform.

Importantly, the media can be designed to select for bacteria of interest. In one embodiment, lactose is added such that lactose-metabolizing bacteria change the pH of the media, but other types of bacteria do not. At the same time, bile salts may be added to prevent the growth of lactose-metabolizing bacteria like Lactococcus that are not of interest, resulting in selectivity for fecal coliforms.

The present invention may also be used to select and measure Staphylococcus bacteria, for example, for hospital disposables. To select for Staphylococcus bacteria, the system of the present invention may be used with the substitution of a mannitol salt medium that includes mannitol and sodium chloride.

For coliform-specific media and the mannitol media, a nutrient broth is also included. This can be any number of standard growth mixes. A standard nutrient broth is, for example, 3 g/L of beef extract plus 5 g/L of peptone.

The sensor has at least two electrodes made of a suitably conductive material, for example, a metal or conductive polymer. The electrical sensor is optionally an ohmmeter and is used in an ohmmeter circuit. The ohmmeter circuit can be a voltage divider circuit with a reference resistor. The impedance of the reference resistor should be roughly equivalent to the resistance of the sealable vessel when filled with media, and therefore should be selected based on the size of the sealable vessel and basal conductance of the media. The sealable vessel can be of any suitable size that can be manipulated to incubate a water sample, for example, from tens of micrometers to tens of centimeters. However, small numbers of bacteria will be easier to detect in smaller sealable vessels. In one embodiment, the sealable vessel is a two centimeter by two centimeter by one centimeter acrylic box. In another embodiment, the sealable vessel comprises two 3D-printed saucers, each about five centimeters in diameter and one half centimeter high. The two saucers are sealed together to form the sealable vessel. For the acrylic box, a five to 22 kilo-ohm resistor is used. For the 3D-printed saucers, a 15 to 78 kilo-ohm resistor is used.

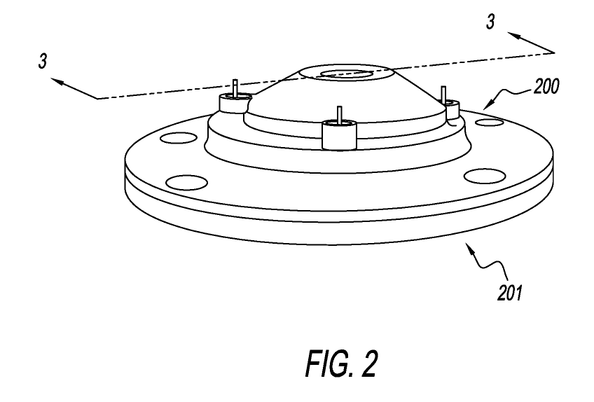

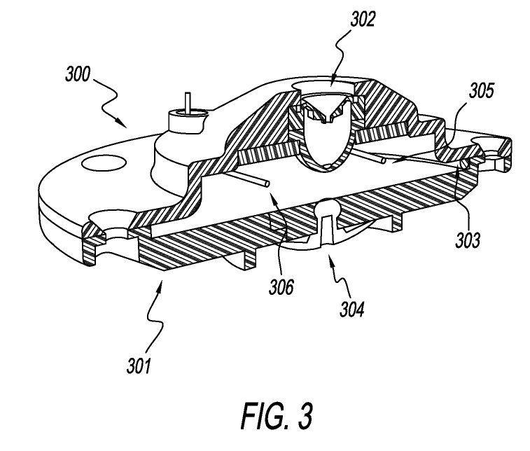

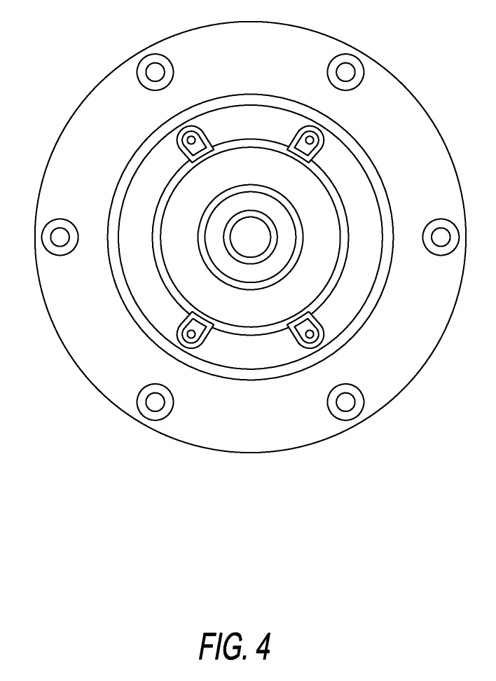

FIGS. 2 through 4 show one embodiment of the sealable vessel. FIG. 2 shows an overhead view of two 3D-printed saucers, with the top saucer, 200, attached to the bottom saucer, 201. FIG. 3 shows a cross-section of the same device, with the top saucer, 300, still shown sealed to the bottom saucer, 301. Both the bacterial sample and selective media can be flown into the internal cavity of the sealed vessel via a one-way ingress valve, 302. Dilute bacterial sample is passed over a filter within the vessel, 303. This sub-micron filter captures any bacteria in the sample, and excess liquid flows through the egress valve, 304. Bacterial growth media is added to the vessel via the ingress valve, 302, and bacteria trapped by the filter is incubated for the necessary amount of time within the vessel. The two electrodes exposed within the cavity of the vessel, 305 and 306, pass to the outside, where a sensor component, such as the circuit seen in FIG. 1, measures the relevant electrical property of the media.

A sample of bacteria or, alternatively, a sample of water from a water source can be introduced into the bacterial growth medium. The introduction of the solution of bacteria or the sample of water serves as the inoculum of bacteria to test for concentration using the bacterial concentration measurement system of the present invention.

The sample of water collected from the water source may be sampled for one or more collection times to allow periodic or continuous sampling. Each of the solution of bacteria or the sample of water will be separately incubated. The solution of bacteria or the sample of water are incubated at an incubation temperature and for an incubation time which are appropriate or optimal for growth of the species of the selected bacteria.

During the incubation, the selected metabolism of the bacterial growth medium by the selected bacteria results in the change in the physico-chemical properties of the bacterial growth medium that further results in changes in electrical properties of the bacterial growth medium. The electrical sensor is in contact with the bacterial growth medium and detects and monitors the changes in electrical properties of the bacterial growth medium as the measurement of the standard SI unit of electricity during the incubation time. The concentration of the selected bacteria in the sample of water or solution of bacteria is calculated from the difference in measurements of a standard SI unit of electricity in the bacterial growth medium over the incubation time.

In another embodiment of the present invention, the changes in electrical properties of the bacterial growth medium caused by the metabolites of the bacterial growth medium is a change in electrical resistance and the standard SI units of electricity that is measured with the electrode is ohms.

In another embodiment of the present invention, the change in physico-chemical properties of the bacterial growth medium from the metabolites is a change in pH.

In another embodiment of the present invention, the sealable vessel may be formed of a rigid or semi-rigid material.

In another embodiment of the present invention, the rigid or semi-rigid material is a plastic.

In another embodiment of the present invention, the electrical sensor further comprises electrodes that are connected to a voltage-divider circuit that measures the resistance of an unknown resistor with respect to a reference resistor in order to produce the measurement of electrical resistance of the bacterial growth medium.

In another embodiment of the present invention, the electrodes comprise exposed copper leads.

In another embodiment of the present invention, the voltage-divider circuit is powered with an AC square wave-form voltage current or a DC voltage current, in which DC voltage current, the direction of power is switched across the electrodes to avoid polarization of the bacterial growth medium.

In another embodiment of the present invention, the selected bacteria are of species in the genus Enterococcus.

In another embodiment of the present invention, the selected bacteria are of species in the genus Escherichia.

In another embodiment of the present invention, the growth medium contains bile constituents selected from the group consisting of a salt of deoxycholic acid, a salt of cholic acid, bovine bile, or ox bile.

In another embodiment of the present invention, the bile constituents are at a concentration of from about 0.6 grams to about 3 grams per liter of bacterial growth media.

In another embodiment of the present invention, the incubation temperature is in a range of from about 30 degrees centigrade to about 40 degrees centigrade.

In another embodiment of the present invention, the incubation temperature may be more narrowly specified. For example, in a range of from about 36.5 to about 37.5 degrees centigrade.

In another embodiment of the present invention, the incubation temperature is selected to take advantage of a specific range of temperature tolerance for the selected bacteria to further enhance preferential selection. For example, the incubation temperature for E. coli may be in a range of from about 60 degrees centigrade to about 70 degrees centigrade.

In another embodiment of the present invention, the collection time is from about 4 to about 24 hours.

In another embodiment of the present invention, the collection time is from about 8 to about 12 hours.

In another embodiment of the present invention, the incubation time is from about 6 to about 48 hours.

In another embodiment of the present invention, the incubation time is from about 12 to about 16 hours.

The variations in the elements of the invention will be readily understood by one of skill in the art to be interchangeable with the elements described herein. These descriptions of the sensor element variations in the present invention are intended to be exemplary and are not intended to be limiting in any way.

### Example I

A test for detection and measurement of E. coli bacteria using the present invention was made. A sealable vessel made of a thermoplastic resin having a volume over 1. milliliter was prepared. Two copper electrodes were inserted into the sealable vessel so that they do not touch and also so that one end of each electrode protrudes from the sealable vessel. The electrodes were fitted to the vessel such that, when filled, they are partly submerged in the medium. The ends of the electrodes that are not submerged were attached to an ohmmeter to measure the resistance of the medium contained in the sealable vessel when filled.

Once prepared, the sealable vessel was filled with growth medium. To select for the fecal coliform, E. coli, a lactose and bile salt medium was used, using the following recipe: 10 g. lactose, 5 g. peptone, 3 g. beef extract, and 1.5 g. bile salts added to 1 Liter of water and dissolved.

The bacterial sample or water sample was then placed into the medium. Since this was a small volume of sample, the sample was mixed directly with the medium. For larger volumes, that would significantly dilute the medium, the sample solution can be filtered through a 450 nanometer filter. The filter with collected bacteria was then placed directly into the medium, ensuring that the filter itself does not contact the electrodes. For bacteria in non-liquid environments, for example, food or soil, standard bacterial extraction methods such as homogenization followed by dissolving and filtering can be used.

The sealable vessel containing the medium and the sample was then placed in an incubation chamber at 65 degrees C. to encourage bacterial growth. For most other coliforms, about 37 degrees C. is sufficient.

The circuit illustrated in FIG. 1 was then turned on, and resistance was measured continuously across the vessel. The circuit was a voltage divider circuit with a 39 kOhm reference resistor and 5V power source. Every 500 milliseconds, the circuit made a reading of the voltage that passed through the vessel from the power source and derived the resistance of the vessel based on a known reference resistor. The circuit switched the direction of the current for each reading so that the metabolites and other constituents in the medium in the vessel were not polarized or gathered along one electrode. This imitates the effect of an AC square wave current using a simple DC power source. Thus, two readings were taken about every second. These can be treated separately or averaged together.

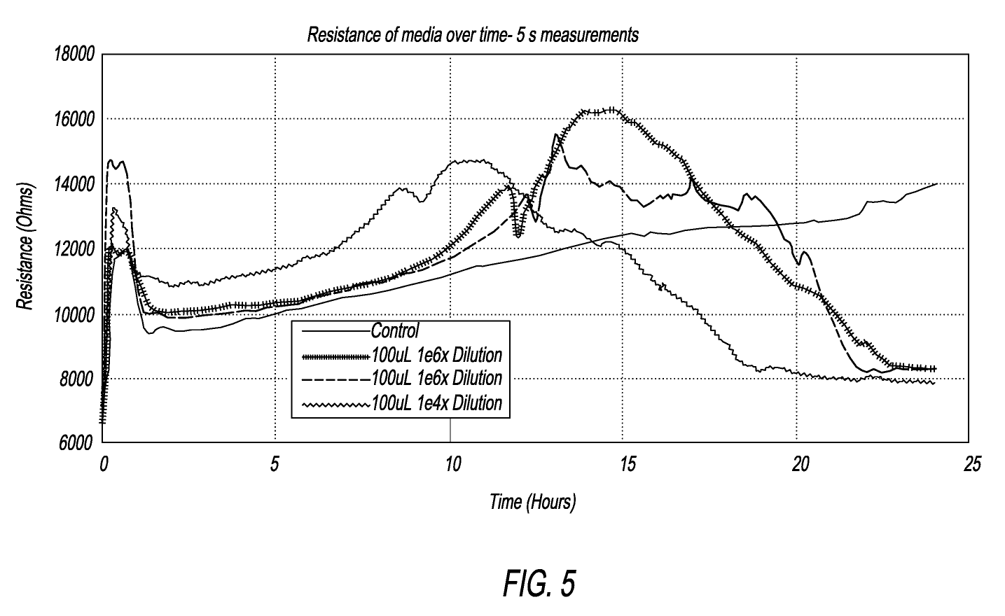

The circuit ran in this manner for 24 hours. If no target bacteria are present, the resistance of the media settles over the first hour, and then gradually increases over the course of the incubation. If the bacteria of interest are present, they will replicate in the nutrient broth and begin to metabolize the metabolites included in the medium, resulting in a change in the pH of the media. In the case of lactose metabolization, an initial spike in resistance was observed as the bacteria grew, then a gradual decrease in resistance of the medium occurred as the pH decreased and the stoichiometry of ions in the media changed as shown in FIG. 5.

Using the resistance traces from this circuit, we determined if the target bacteria, E. coli, was present in the incubation medium. Assuming that temperature is consistent across samples, the nature of the curve can be used to determine relative bacterial concentration, as the time between starting the incubation and the initial peak in resistance of the media reflects the total concentration of bacteria in the sample.

### Example 2

Alternatively, to use the present invention for selection and measurement of Enterococci bacteria, the same system set-up may be used with the modification of medium that is selective for the particular bacteria of interest.

For the detection of nitrifying bacteria in sewage treatment water samples, the chemical composition of the medium used is 0.5 (NH4)2SO4; 1.0 K2HPO4; 0.03 FeSO4.7 H20; 0.3 NaCl; 0.3 MgSO4.7 H2O; and 7.5 CaCO3.

### Example 3

To use the present invention for selection and measurement of Staphylococcus bacteria, for example, for hospital disposables, the same system set-up may be used with the modification of medium using a mannitol salt media including 75 g/L NaCl and 10 g/L mannitol.

We claim:

1. A bacterial concentration measurement system comprising: a sealable vessel having an electrical sensor that can produce measurements of a standard SI unit of electricity, is contained therein,
wherein the sealable vessel contains bacterial growth medium,
wherein the bacterial growth medium comprises lactose and bile constituents to enable the growth of selected bacteria which are able to metabolize the lactose and tolerate the bile constituents,
wherein, into the bacterial growth medium a solution of bacteria or a sample of water collected from a water source for a collection time are introduced,
wherein the solution of bacteria or the sample of water are incubated at an incubation temperature in the bacterial growth medium for an incubation time,
wherein the metabolism of the bacterial growth medium by the selected bacteria results in a change in physico-chemical properties of the bacterial growth medium,
wherein the change in the physico-chemical properties of the bacterial growth medium further results in changes in electrical properties of the bacterial growth medium,
wherein the electrical sensor is in contact with the bacterial growth medium and detects the changes in electrical properties of the bacterial growth medium as the measurement of the standard SI unit of electricity,
wherein the measurements of a standard SI unit of electricity are monitored during the incubation time, and
wherein the concentration of the selected bacteria in the sample of water or solution of bacteria is calculated from the measurements of a standard SI unit of electricity in the bacterial growth medium over time.

2. The bacterial detection system of claim 1, further wherein the changes in electrical properties of the bacterial growth medium is a change in electrical resistance and the standard SI units of electricity are ohms.

3. The bacterial detection system of claim 2, wherein the change in physico-chemical properties of the bacterial growth medium is a change in pH.

4. The bacterial detection system of claim 1, further wherein water from an exogenous source may be introduced for a collection time in order to test for the presence of bacteria.

5. The bacterial detection system of claim 1, wherein the sealable vessel may be formed of a rigid or semi-rigid material.

6. The bacterial detection system of claim 5, wherein the rigid or semi-rigid material is a plastic.

7. The bacterial detection system of claim 2, wherein the electrical sensor further comprises, electrodes that are connected to a voltage-divider circuit that measures the resistance of an unknown resistor with respect to a reference resistor in order to produce the measurement of electrical resistance of the bacterial growth medium.

8. The bacterial detection system of claim 7, wherein the electrodes comprise exposed copper leads.

9. The bacterial detection system of claim 7, wherein the voltage-divider circuit is powered with an AC square wave-form voltage current or a DC voltage current, in which DC voltage current, the direction of power is switched across the electrodes to avoid polarization of the bacterial growth medium.

10. The bacterial detection system of claim 1, wherein the selected bacteria are of species in the genus Enterococcus.

11. The bacterial detection system of claim 1, wherein the selected bacteria are of species in the genus Escherichia.

12. The bacterial detection system of claim 1, wherein the bile constituents are selected from the group consisting of a salt of deoxycholic acid, a salt of cholic acid, bovine bile, or ox bile.

13. The bacterial detection system of claim 12, wherein the bile constituents are at a concentration of from about 0.6 grams to about 3 grams per liter of bacterial growth media.

14. The bacterial detection system of claim 1, further comprising, where the incubation temperature is in a range of from about 30 degrees centigrade to about 40 degrees centigrade.

15. The bacterial detection system of claim 1, further comprising wherein the incubation temperature is in a range of from about 60 degrees centigrade to about 70 degrees centigrade.

16. The bacterial detection system of claim 1, wherein the collection time is from about 4 to about 24 hours.

17. The bacterial detection system of claim 16, wherein the collection time is from about 8 to about 12 hours.

18. The bacterial detection system of claim 17, wherein the incubation time is from about 6 to about 48 hours.

19. The bacterial detection system of claim 18, wherein the incubation time is from about 12 to about 16 hours.
